(19) United States
(12) Patent Application Publication
(10) Pub. No.: US 2019/0105274 A1
(43) Pub. Date: Apr. 11, 2019

(54) TABLETS CONTAINING ARGININE AT HIGH CONCENTRATION

(71) Applicant: KYOWA HAKKO BIO CO., LTD., Tokyo (JP)

(72) Inventors: Takuya ASAOKA, Tokyo (JP); Yuichiro NAKANO, Tokyo (JP); Yoshiyuki FUJIMORI, Tokyo (JP)

(73) Assignee: KYOWA HAKKO BIO CO., LTD., Tokyo (JP)

(21) Appl. No.: 15/743,138

(22) PCT Filed: Jul. 12, 2016
(86) PCT No.: PCT/JP2016/070601

§ 371 Date: Jan. 9, 2018

(30) Foreign Application Priority Data
Jul. 13, 2015 (JP) 2015-139813

(51) Int. Cl. A61K 9/20; A61K 31/198

(52) U.S. Cl. CPC A61K 9/2054; A61K 9/2095; A61K 9/2072; A61K 9/2009; A61K 31/198; A61K 9/2013

(57) ABSTRACT

The invention provides a tablet that contains free-form arginine at a high content, suppresses cracking and collapse due to moisture absorption, is superior in preservation stability and permits convenient production. The tablet contains free-form arginine at a concentration of not less than 5 mass % of the total amount of the tablet and can be obtained by compression molding free-form arginine dried by a spray dry method.

### TECHNICAL FIELD

The present invention relates to a tablet containing free-form arginine at a high concentration and superior in preservation stability, and a production method of the tablet.

### BACKGROUND ART

Since arginine has an effect of increasing basal metabolism such as promotion of growth hormone secretion, improvement of blood flow, and the like, it is commercially available as a supplement and the like. As commercially available arginine preparations, capsules and granular formulations are known. However, ingestion of an amount expected to provide effects in a capsule or granular formulation has the following problems.

The problem of capsule is that large capsules must be ingested in large amounts since a compression process is not applied to capsules, and the problems of granules are that the taste and smell of arginine are annoying and packaging materials are costly and the like.

Accordingly, as a form for ingestion of arginine, tablet is preferable.

However, it is known as regards tablets containing arginine that a tablet containing a given amount or more of free-form arginine relative to the total amount of the tablet cracks or collapses under humidified conditions. In an attempt to avoid such cracking and collapsing, a technique including wet-granulating a powder containing arginine to give a granulated product containing 2.7% by mass or more of moisture, followed by tableting has been reported (patent document 1).

On the other hand, when glutamate of arginine is contained in a tablet, occurrence of crack and collapse under humidified conditions can be suppressed. Therefore, for example, KYOWA HAKKO BIO CO., LTD. sells tablets containing arginine glutamate.

However, tablets containing arginine glutamate contain arginine and glutamic acid at almost the same ratio, which limits the arginine content of the tablet. That is, a large amount of tablets need to be ingested to ingest arginine in an amount expected to provide effects.

In addition, a technique for covering granules containing water-absorbing amino acid such as arginine or the like with an ethanol-soluble and water poorly-soluble coating agent has been reported (patent document 2). Moreover, a technique for coating granules containing a drug unstable to water and the like to give a solid preparation, and a technique for coating a core granule with a sugar coating solution has been reported (patent documents 3, 4). When a coating is applied, however, problems of prolonged production time and increased production costs occur, even though advantages such as masking of undesirable taste and smell and superior storage stability are afforded.

Under such circumstances, a tablet containing free-form arginine at a high concentration, permitting convenient production and superior in preservation stability has been desired.

### DOCUMENT LIST

### Patent Documents

patent document 1: JP-A-2010-254580
patent document 2: JP-A-2005-298373
patent document 3: JP-A-2007-001873
patent document 4: JP-A-2007-197378

### SUMMARY OF THE INVENTION

### Problems to be Solved by the Invention

Accordingly, the present invention aims to provide a tablet containing free-form arginine at a high concentration, permitting convenient production and superior in preservation stability, and a production method thereof.

### Means of Solving the Problems

The present inventors have conducted intensive studies in an attempt to solve the above-mentioned problems and found that a tablet containing free-form arginine at a high concentration can be obtained by compression molding free-form arginine dried by a spray dry method, and that crack and collapse due to moisture absorption during preservation is suppressed in the tablet, which resulted in the completion of the present invention.

Accordingly, the present invention relates to the following [1]-[4].

[1] A tablet containing free-form arginine in a proportion of not less than 5 mass % of the total amount of the tablet.
[2] The tablet of the above-mentioned [1], wherein the arginine is L-arginine.
[3] A method of producing a tablet containing free-form arginine, comprising a step of drying a water mixture of the free-form arginine by a spray dry method, and a step of compression molding the obtained dry product.
[4] The production method of the above-mentioned [3], wherein the arginine is L-arginine.

### Effect of the Invention

The present invention provides a tablet containing free-form arginine in a proportion of not less than 5 mass % of the total amount of the tablet, suppressing crack or collapse due to moisture absorption, and superior in preservation stability.

In addition, the tablet of the present invention can be produced conveniently because it does not require a coating step such as one for conventional coated preparations.

Furthermore, the tablet of the present invention permits miniaturization of tablet because it contains free-form arginine at a high concentration.

### BRIEF DESCRIPTION OF THE DRAWINGS

FIG. 1 shows the state of the upper surface of the tablet produced in Example 1, after preservation at 40° C., relative humidity 75% for 24 hr.

FIG. 2 shows the state of the side surface of the tablet produced in Example 1, after preservation at 40° C., relative humidity 75% for 24 hr.

FIG. 3 shows the state of the upper surface of the tablet produced in Example 2, after preservation at 40° C., relative humidity 75% for 24 hr.

FIG. 4 shows the state of the side surface of the tablet produced in Example 2, after preservation at 40° C., relative humidity 75% for 24 hr.

FIG. 5 shows the state of the upper surface of the tablet produced in Comparative Example 1, after preservation at 40° C., relative humidity 75% for 1 hr.

FIG. 6 shows the state of the side surface of the tablet produced in Comparative Example 1, after preservation at 40° C., relative humidity 75% for 1 hr.

FIG. 7 shows the state of the upper surface of the tablet produced in Comparative Example 2, after preservation at 40° C., relative humidity 75% for 2 hr.

FIG. 8 shows the state of the side surface of the tablet produced in Comparative Example 2, after preservation at 40° C., relative humidity 75% for 2 hr.

### DESCRIPTION OF EMBODIMENTS

### (Tablet of the Present Invention)

The present invention is a tablet containing free-form arginine in a proportion of not less than 5 mass % of the total amount of the tablet (hereinafter to be referred to as the tablet of the present invention).

Arginine (5-guanidino-2-aminopentanoic acid) is an amino acid showing basicity. In the tablet of the present invention, a free form, namely, arginine in a free form, which does not form a salt, is used.

In the tablet of the present invention, as arginine, one produced by a production method known per se such as a method including extraction and separation from acid hydrolysates such as gelatin, defatted soybean and the like, a chemical synthesis method using ornithine as a starting material, and a fermentation method using 2-thiazole alanine resistance+guanine auxotroph strain of Brevibacterium flavum and the like can be used without limitation. One produced by a fermentation method is preferably used.

As the free-form arginine, one produced according to the above-mentioned known production method may be used or, for example, a commercially available product provided by KYOWA HAKKO BIO CO., LTD. and the like may be used.

As arginine, any of D-form, L-form and DL-form can be used, and L-form is preferably used.

In the tablet of the present invention, a water mixture of free-form arginine is dried by a spray dry method and contained in the tablet.

The spray dry method is a method including spraying a solution or suspension of a drug or the like together with hot air from a nozzle with a small pore size and drying same as minute droplets in a short time in a chamber, and can afford spherical powder particles rich in flowability. In the present invention, spray drying can be performed under conditions generally used for formulating.

The water mixture of free-form arginine to be subjected to the above-mentioned spray dry method is an aqueous free-form arginine solution obtained by adding free-form arginine to water and homogeneously mixing same, or a water suspension in which free-form arginine added is partly dissolved in water and the rest is suspended in water. The concentration of arginine added in the water mixture is generally 10 (w/v) %-80 (w/v) %.

The water mixture of free-form arginine is spray dried using, for example, an open-type spray dryer. As the open-type spray dryer, various instruments for pharmaceutical products and foods, which are manufactured and provided by respective companies, can be used, and can be appropriately selected and used according to the production scale, namely, the amount of the water mixture of free-form arginine to be subjected to a spray drying treatment and the like. Examples of the open-type spray dryer include L-8i-model spray dryer, OC-16-model spray dryer and OC-20-model spray dryer (all manufactured by OHKAWARA KAKOHKI CO., LTD.).

In drying a water mixture of free-form arginine by a spray dry method, the properties of the obtained powder particles can be controlled by adjusting the inlet temperature and outlet temperature of the spray dryer, selection of an atomizer to be used and the like.

The inlet temperature is preferably 80° C.-200° C., more preferably 100° C.-180° C. The outlet temperature is preferably 40° C.-85° C., more preferably 50° C.-70° C.

The atomizer for spray dryer includes one type that performs atomization by the energy of an air stream and another type that performs atomization by a centrifugal force.

The type for performing atomization by the energy of an air stream air includes gas blast atomizer such as air blast atomizer and the like, gas assist atomizer such as air assist atomizer and the like, effervescent atomizer and the like. A gas-liquid contact method therefor includes prefilming type, plain-jet type, cross-flow type and the like, and a gas-liquid mixing method therefor includes external mixing type, internal mixing type, Y-jet type and the like. Representative ones include prefilming air blast atomizer, plain-jet air blast atomizer, external mixing air assist atomizer, internal mixing air assist atomizer, Y-jet atomizer and the like.

The type that performs atomization by a centrifugal force includes rotary cup atomizer, rotary disc atomizer, wheel atomizer and the like. A rotary disc atomizer such as M-type disc atomizer, rotary disc atomizer or the like is preferably used, and rotation number thereof is preferably 25,000 rpm-40,000 rpm.

An arginine powder with improved sphericity can be obtained by drying a water mixture of free-form arginine by a spray dry method.

The moisture content of an arginine powder obtained by drying by a spray dry method, which is measured by a heating and drying type moisture analyzer method or Karl Fischer's method, is not more-than 5.5 mass %, preferably not more than 4.5 mass %, more preferably not more than 4 mass %.

The tablet of the present invention contains the above-mentioned powder of free-form arginine obtained by drying by a spray dry method. The tablet of the present invention contains the powder in a proportion of, based on the amount of free-foLm arginine free of water, not less than 5 mass %, preferably not less than 10 mass %, more preferably not less than 20 mass %, yet preferably not less than 33 mass %, yet more preferably not less than 37 mass %, further more preferably not less than 47 mass %, yet further more preferably not less than 56 mass %, yet still preferably not less than 66 mass %, still further more preferably not less than 75 mass %, particularly preferably not less than 85 mass %, most preferably not less than 87 mass %, relative to the total amount of the tablet.

In consideration of the preparation stability and the like of the tablet, the upper limit of the content thereof in the tablet of the present invention, based on the amount of free-form arginine free of water, is not more than 99 mass %, preferably not more than 98 mass %, more preferably not more than 96 mass %, relative to the total amount of the tablet.

The tablet of the present invention can also contain additives generally used for formulating such as excipient, binder, disintegrant, fluidizer, lubricant, preservative, antioxidant, colorant, corrigent, souring agent and the like. These additives are contained as necessary in the tablet of the present invention according to a general method for the production of tablets as long as the characteristics of the present invention are not impaired.

Examples of the excipient that can be contained in the tablet of the present invention include lactose, sucrose, D-mannitol, D-sorbitol, cornstarch, dextrin, carboxymethylcellulose, carboxymethylcellulose calcium, sodium carboxymethyl starch, crystalline cellulose and the like.

Examples of the binder that can be contained in the tablet of the present invention include hydroxypropylcellulose, hydroxypropylmethylcellulose, polyvinylpyrrolidone, sucrose, dextrin, starch, pregelatinized starch, gelatin, carboxymethylcellulose sodium, gum arabic and the like.

Examples of the disintegrant that can be contained in the tablet of the present invention include carboxymethylcellulose, carboxymethylcellulose calcium, carboxymethylcellulose sodium, sodium carboxymethyl starch, croscarmellose sodium, crospovidone, glycerol fatty acid ester and the like.

Examples of the fluidizer that can be contained in the tablet of the present invention include tricalcium phosphate, light anhydrous silicic acid, magnesium stearate and the like.

Examples of the lubricant that can be contained in the tablet of the present invention include magnesium stearate, calcium stearate, sodium stearyl fumarate, stearic acid, talc and the like.

Examples of the preservative that can be contained in the tablet of the present invention include methyl p-hydroxybenzoate, sodium dehydroacetate, D-sorbitol and the like.

Examples of the antioxidant that can be contained in the tablet of the present invention include sodium sulfite, tocopherol acetate, natural vitamin E and the like.

Examples of the colorant that can be contained in the tablet of the present invention include food colors (e.g., Food Color Red No. 2 or 3, Food Color Yellow No. 4 or 5 etc.), carotene and the like.

Examples of the corrigent that can be contained in the tablet of the present invention include saccharin sodium, dipotassium glycyrrhizinate, aspartame and the like.

Examples of the souring agent that can be contained in the tablet of the present invention include citric acid, malic acid, phosphoric acid, fumaric acid and the like.

In the tablet of the present invention, a tablet superior in the preservation stability can be obtained even when a coating treatment is not applied, as mentioned below. However, a coating treatment with various coating materials may be applied to impart functions such as masking of taste and smell, enteric property and the like. For example, sucrose and the like may be applied to give sugar-coated tablets, hydroxypropylmethylcellulose phthalate, cellulose acetate phthalate, methacrylic acid copolymer (Eudragit L100, Eudragit S100 etc., manufactured by Evonik) and the like may be applied to give enteric tablets, or aminoalkyl methacrylate copolymer (Eudragit RL100, Eudragit RS100 etc., manufactured by Evonik), ethylcellulose and the like may be applied to give sustained release tablets.

The tablet of the present invention can be produced by directly compression molding the above-mentioned powder of free-form arginine that is obtained by drying by a spray dry method, or adding the above-mentioned general additives for preparations such as excipient, binder, disintegrant and the like to the powder as necessary, mixing them to homogeneity and directly compression molding the mixture.

### (Production Method of the Tablet of the Present Invention)

The present invention also provides a production method of the tablet of the present invention. The production method of the tablet of the present invention is a production method of a tablet containing free-form arginine, including a step of drying a water mixture of the free-form arginine by a spray dry method, and a step of compression molding the obtained dry product (hereinafter to be referred to as the production method of the present invention).

As arginine, any of D-form, L-form and DL-form can be used, and L-form is preferably used.

The method of drying an aqueous solution of free-form arginine by a spray dry method is as mentioned above.

A dry product obtained by drying a water mixture of free-form arginine by a spray dry method can be obtained as a free-form arginine powder with high sphericity, as mentioned above.

In the production method of the present invention, the above-mentioned general additives for preparations such as excipient, binder, disintegrant and the like. are added to such dry product of free-form arginine as necessary, the mixture is homogeneously mixed and directly compression molded to give tablets.

In the production method of the present invention, a step of granulating by a stirring granulation method, a fluid bed granulation method, a kneading granulation method or the like may be performed before compression molding. However, a tablet superior in the preservation stability can be produced even without such granulation step.

In the production method of the present invention, moreover, a tablet superior in the preservation stability can be produced without including a step of a coating treatment of free-form arginine or a dried product thereof by a spray dry method with various coating materials.

As used herein, “without including a step of a coating treatment of free-form arginine or a dried product thereof by a spray dry method” means that free-form arginine, a free-form arginine powder obtained by drying by a spray dry method, and granules thereof are not subjected to a coating treatment before performing compression molding.

A dried product of the above-mentioned water mixture of free-form arginine by a spray dry method can be mixed with general additives such as excipient and the like according to a method generally used for formulating and, for example, various mixers, mixing and stirring machines and the like, such as a horizontal cylindrical type mixer, a V-type mixer, a double cone type mixer, an orbital rotary type mixer, a single screw ribbon type mixer, a multiple screw paddle type mixer, a tumbling type mixer, a cone screw type mixer and the like can be used for the mixing.

The above-mentioned dried product of a water mixture of free-form arginine by a spray dry method, and a mixture of the dried product and general additives such as excipient and the like can be compression molded according to a method generally used for formulating and, for example, using a vertical molding machine, a rotary molding machine and the like.

The compression molding pressure (tabletting pressure) is preferably 500 kgf-3,000 kgf, more preferably 600 kgf-2,800 kgf.

The tablet obtained by the production method of the present invention generally has a hardness of 4 kgf-20 kgf as measured by a tablet breaking-strength tester.

The tablet obtained by the production method of the present invention contains free-form arginine at a high concentration, is superior in the preparation strength and preservation stability, suppresses crack and collapse due to moisture absorption, and is also superior in the impact resistance.

Containing free-form arginine at a high concentration, it is possible to pursue miniaturization of the tablet. In addition, since dosing frequency necessary for ingesting an effective amount of arginine can be reduced, the tablet is advantageous in medication compliance.

The tablet of the present invention can be preferably administered orally to mammals such as human, monkey, horse, bovine, sheep, goat, swine, dog, cat, rat, mouse, guinea pig and the like, and can be administered for the purpose of the treatment of congenital urea cycle disorder, as well as promotion of growth hormone secretion, improvement of blood flow, and increase of base metabolism: It can also be ingested as food with health claims such as food for specified health uses, food with nutrient function claims, foods with function claims and the like, health aid food, supplement or the like, with the hope of achieving effects such as improvement of blood flow, increase of base metabolism and the like.

The dose of the tablet of the present invention varies depending on the animal species, sex, age, level of disease or symptom and the like, and can be adjusted by appropriately increasing or decreasing. In the case of a human (adult) with body weight 60 kg, the dose in the amount of free-form arginine is generally 200 mg/day-10,000 mg/day, preferably 400 mg/day-6,000 mg/day, which may be ingested at once or in several divided portions.

While the present invention is explained in more detail in the following by referring to Examples, the present invention is not limited by the following Examples.

In the following Reference Examples, Examples and Comparative Examples, product name “L-arginine KYOWA” (manufactured by KYOWA HAKKO BIO CO., LTD.) was used as L-arginine, product name “CEOLUS UF-F702” (manufactured by Asahi Kasei Chemicals Corporation) was used as crystalline cellulose, product name “tricalcium phosphate” (manufactured by Taihei Chemical Industrial Co., Ltd.) was used as tricalcium phosphate, product name “CELNY-SSL-SFP” (manufactured by NIPPON SODA CO., LTD.) was used as hydroxypropylcellulose, and product name “POEM TR-FB” (manufactured by RIKEN VITAMIN CO., LTD.) was used as glycerol fatty acid ester

### Reference Example 1

600 g of L-arginine was added to 5 L of water, and the mixture was mixed, and spray dried by a spray dryer [L-8i type spray dryer (manufactured by OHKAWARA KAKOHKI CO., LTD.)] set to the conditions of atomizer rotation number=35,000 rpm, inlet temperature=180° C. and outlet temperature=70° C. to give a powder. The moisture content of the aforementioned powder was measured by a heating and drying method moisture analyzer [heating and drying method moisture analyzer MX-50 (manufactured by A&D Company, Limited)] to find 1.96 mass %. The moisture content was 2.3 mass % when measured by the Karl Fischer's method at the General Incorporated Foundations Japan Food Research Laboratories. Hereinafter this powder is indicated to as “SD arginine-A”.

### Reference Example 2

600 g of L-arginine was added to 5 L of water, and the mixture was mixed, and spray dried by a spray dryer [L-8i type spray dryer (manufactured by OHKAWARA KAKOHKI CO., LTD.)] set to the conditions of atomizer rotation number=35,000 rpm, inlet temperature=130° C. and outlet temperature=60° C. to give a powder. The moisture content of the aforementioned powder was measured by a heating and drying method moisture analyzer [heating and drying method moisture analyzer MX-50 (manufactured by A&D Company, Limited)] to find 1.81 mass %. Hereinafter this powder is indicated to as “SD arginine-B”.

### Reference Example 3

600 g of L-arginine was added to 5 L of water, and the mixture was mixed, and spray dried by a spray dryer [L-81 type spray dryer (manufactured by OHKAWARA KAKOHKI CO., LTD.)] set to the conditions of atomizer rotation number=35,000 rpm, inlet temperature=100° C. and outlet temperature=50° C. to give a powder. The moisture content of the aforementioned powder was measured by a heating and drying method moisture analyzer [heating and drying method moisture analyzer MX-50 (manufactured by A&D Company, Limited)] to find 3.80 mass %. The moisture content was 3.91 mass % when measured by the Karl Fischer's method at the General Incorporated Foundations Japan Food Research Laboratories. Hereinafter this powder is indicated to as “SD arginine-C”.

### Example 1

80 g of SD arginine-A, 16 g of crystalline cellulose, 1 g of tricalcium phosphate and 3 g of glycerol fatty acid ester were mixed, compression molded using a compression molding machine (one-shot-type) [vertical molding machine 6B-2M (manufactured by Kikusui Seisakusho Ltd.)] at compression molding pressure=1,500 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Example 2

96 g of SD arginine-A, 1 g of tricalcium phosphate and 3 g of glycerol fatty acid ester were mixed, compression molded using a compression molding machine (one-shot-type) [vertical molding machine 6B-2M (manufactured by Kikusui Seisakusho Ltd.)] at compression molding pressure=2,200 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Example 3

93 g of SD arginine-A, 3 g of hydroxypropylcellulose, 1 g of tricalcium phosphate and 3 g of glycerol fatty acid ester were mixed, compression molded using a compression molding machine (one-shot-type) [vertical molding machine 6B-2M (manufactured by Kikusui Seisakusho Ltd.)] at compression molding pressure=1,600 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Example 4

35 g of SD arginine-A, 61 g of crystalline cellulose, 1 g of tricalcium phosphate and 3 g of glycerol fatty acid ester were mixed, compression molded using a compression molding machine (one-shot-type) [vertical molding machine 6B-2M (manufactured by Kikusui Seisakusho Ltd.)] at compression molding pressure=650 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO.; LTD.)]. As a result, the tablet hardness was 10 kgf on average.

### Example 5

96 g of SD arginine-B, 1 g of tricalcium phosphate and 3 g of glycerol fatty acid ester were mixed, compression molded using a compression molding machine (one-shot-type) [vertical molding machine 6B-2M (manufactured by Kikusui Seisakusho Ltd.)] at compression molding pressure=2,500 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Example 6

96 g of SD arginine-C, 1 g of tricalcium phosphate and 3 g of glycerol fatty acid ester were mixed, compression molded using a compression molding machine (one-shot-type) [vertical molding machine 6B-2M (manufactured by Kikusui Seisakusho Ltd.)] at compression molding pressure=2,700 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Comparative Example 1

96 g of L-arginine and 19.2 g of crystalline cellulose were fed into a fluid bed granulator [fluid bed granulation coating machine FL-MINI type (manufactured by Freund Corporation)], and 12 g of water was sprayed while flow mixing them. Thereafter, the mixture was dried to give 96 g of granules. The granules were mixed with 1 g of tricalcium phosphate and 3 g of glycerol fatty acid ester, and the mixture was compression molded using a compression molding machine (one-shot-type) [vertical molding machine 6B-2M (manufactured by Kikusui Seisakusho Ltd.)] at compression molding pressure=1,500 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Comparative Example 2

42 g of L-arginine and 73.2 g of crystalline cellulose were fed into a fluid bed granulator [fluid bed granulation coating machine FL-MINI type (manufactured by Freund Corporation)], and 12 g of water was sprayed while flow mixing them. Thereafter, the mixture was dried to give 96 g of granules. The granules were mixed with 1 g of tricalcium phosphate and 3 g of glycerol fatty acid ester, and the mixture was compression molded using a compression molding machine (one-shot-type) [vertical molding machine 6B-2M (manufactured by Kikusui Seisakusho Ltd.)] at compression molding pressure=550 kgf to give tablets with diameter=9 mm, 300 mg/tablet. Any 10 tablets were selected, and the tablet hardness was measured by a tablet breaking-strength tester [tablet breaking-strength tester TH-203CP (manufactured by TOYAMA SANGYO CO., LTD.)]. As a result, the tablet hardness was 10 kgf on average.

### Experimental Example

The respective tablets obtained in Examples 1-6 and Comparative Examples 1, 2 were preserved in a thermostatic tank [CONSTANT LOW TEMPERATURE/HUMIDITY CHAMBER (manufactured by Toyo Seisakusho Kaisha, Ltd.)] set to temperature=40° C., relative humidity=75% under exposure conditions for 24 hr.

The test results are shown with evaluation points of 0-3 according to the following evaluation criteria, and described in Table 1 together with the content percentage (mass %) of each component, tabletting pressure (compression molding pressure) and tablet hardness of respective tablets of Examples and Comparative Examples.

### <Evaluation Criteria>

0: no problem as tablet

1: slight crack on tablet side surface, surface looks fine, does not break even with impact comparable to falling from a height of 1 m, and no problem as tablet

2: crack developed in tablet, easily collapsed with light impact (when dropped from a height of 20 cm)

3: tablet has collapsed

TABLE 1

| | | | | | | | Comparative | Comparative |
|---|---|---|---|---|---|---|---|---|
| | Example 1 | Example 2 | Example 3 | Example 4 | Example 5 | Example 6 | Example 1 | Example 2 |
| SD arginine-A | 80 | 96 | 93 | 35 | — | — | — | — |
| SD arginine-B | — | — | — | — | 96 | — | — | — |
| SD arginine-C | — | — | — | — | — | 96 | — | — |
| L-arginine | — | — | — | — | — | — | 80 | 35 |
| crystalline | 16 | — | — | 61 | — | — | 16 | 61 |
| cellulose | | | | | | | | |
| hydroxypropyl | — | — | 3 | — | — | — | — | — |
| cellulose | | | | | | | | |
| tricalcium | 1 | 1 | 1 | 1 | 1 | 1 | 1 | 1 |
| phosphate | | | | | | | | |
| glycerol | 3 | 3 | 3 | 3 | 3 | 3 | 3 | 3 |
| fatty acid | | | | | | | | |
| ester | | | | | | | | |
| tabletting | 1,500 | 2,200 | 1,600 | 650 | 2,500 | 2,700 | 1,500 | 550 |
| pressure | | | | | | | | |
| (kgf) | | | | | | | | |
| hardness | 10 | 10 | 10 | 10 | 10 | 10 | 10 | 10 |
| (kgf) | | | | | | | | |
| test results | 0 for 12 hr | 0 | 0 | 0 | 0 | 0 | 3 after 1 hr | 2 after 2 hr |
| | 1 after 24 hr | | | | | | | 3 after 3 hr |

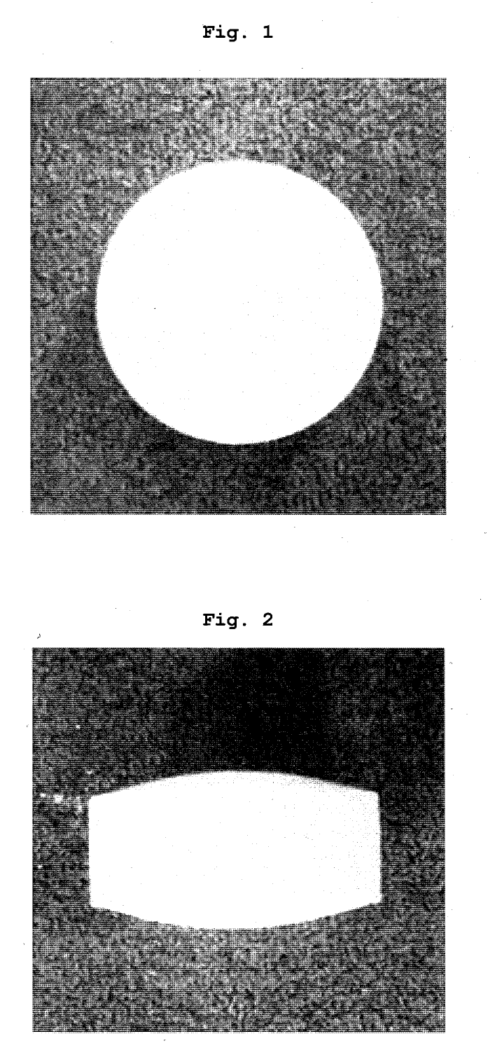

The upper surface and the side surface of the respective tablets produced in Examples 1, 2 and Comparative Examples 1, 2 after performing the above-mentioned test are shown in FIGS. 1-8.

As shown in Table 1, the tablet produced in Example 1 and preserved at 40° C., relative humidity=75% under exposure conditions was evaluated as 0 point up to 12 hr and 1 point after 24 hr.

FIGS. 1, 2 are photographs of the upper surface and the side surface of the tablet produced in Example 1 and preserved at 40° C., relative humidity=75% for 24 hr. The upper surface of the tablet did not have a crack and only a slight crack was found on the side surface.

As shown in Table 1, the tablet produced in Example 2 and preserved at 40° C., relative humidity=75% under exposure conditions was evaluated as 0 point after preservation for 24 hr.

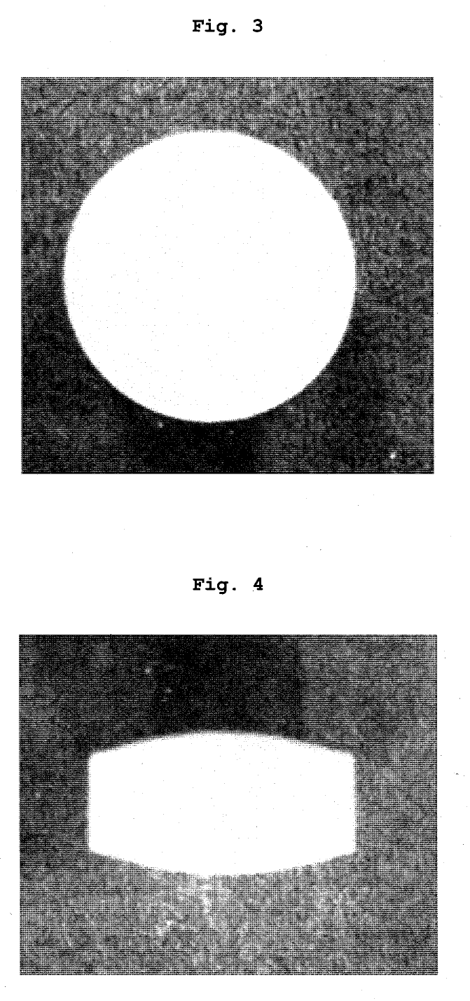

FIGS. 3, 4 are photographs of the upper surface and the side surface of the tablet produced in Example 2 and preserved at 40° C., relative humidity=75% for 24 hr. The surface of the tablet looked fine and even a slight crack was not found on the upper surface and the side surface.

As shown in Table 1, the tablets produced in Examples 3-6 and preserved at 40° C., relative humidity=75% under exposure conditions for 24 hr were respectively evaluated as 0 point. Occurrence of crack due to moisture absorption was not observed, and high preservation stability was found.

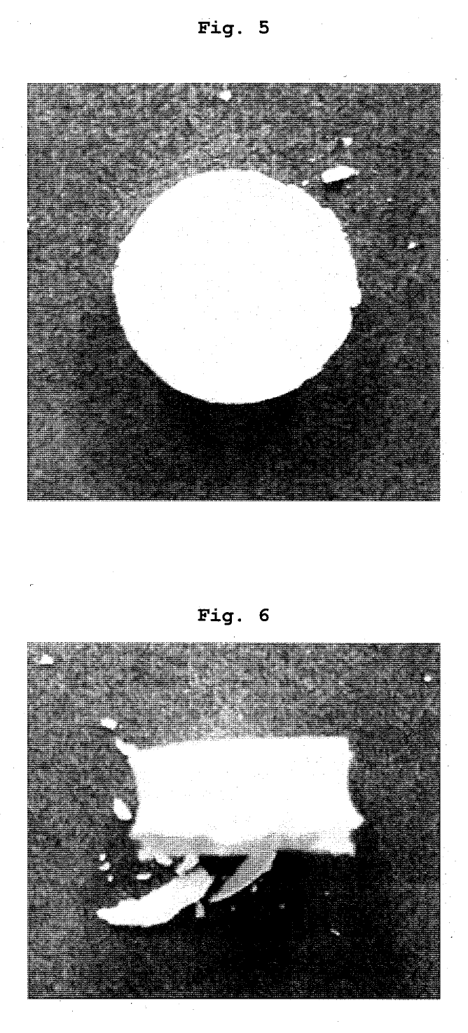

On the other hand, the tablets produced by wet granulating L-arginine together with crystalline cellulose, adding and mixing tricalcium phosphate and glycerol fatty acid ester, and compression molding the mixture (Comparative Examples 1, 2) collapsed in 1 hr-3 hr as shown in Table 1 when they were preserved under exposure conditions at 40° C., relative humidity=75%. The photographs of the upper surface and the side surface of the tablets produced in Comparative Examples 1, 2 and each preserved at 40° C., relative humidity=75% for 1 hr and 2 hr are respectively shown in FIGS. 5-8.

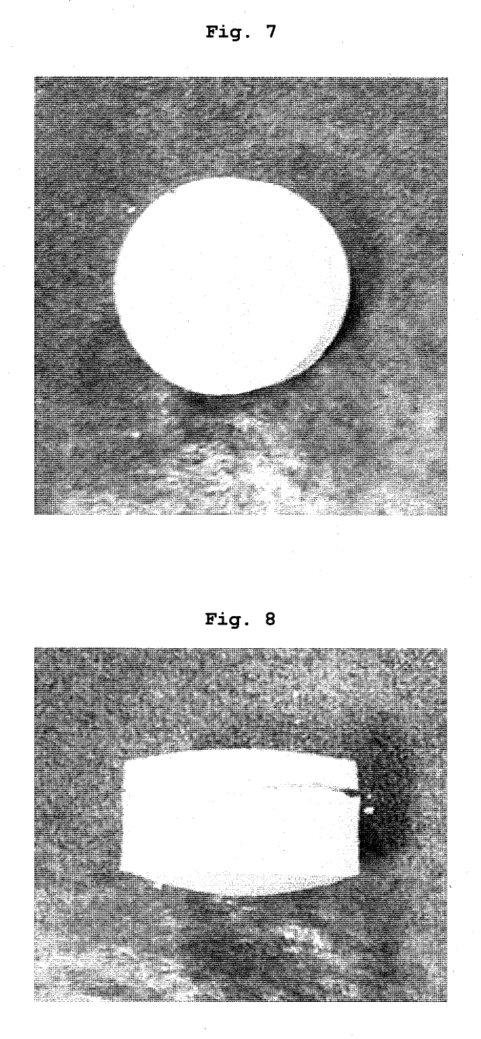

The tablet of Comparative Example 2 having an L-arginine content of 35 mass % showed cracks on the upper surface and the side surface after preservation for 2 hr (FIGS. 7, 8), and the tablet of Comparative Example 1 having an L-arginine content of 80 mass % collapsed after 1 hr (FIGS. 5, 6).

### INDUSTRIAL APPLICABILITY

As described in detail above, the present invention can provide a tablet that contains free-form arginine in a proportion of not less than 5 mass % of the total amount of the tablet, suppresses crack and collapse due to moisture absorption, is superior in preservation stability and permits convenient production.

This application is based on patent application No. 2015-139813 filed in Japan, the contents of which are encompassed in full herein.

1. A tablet comprising free-form arginine in a proportion of not less than 5 mass % of the total amount of the tablet.

2. The tablet according to claim 1, wherein the arginine is L-arginine.

3. A method of producing a tablet containing free-form arginine, comprising a step of drying a water mixture of the free-form arginine by a spray dry method, and a step of compression molding the obtained dry product.

4. The production method according to claim 3, wherein the arginine is L-arginine.
